(19) United States
(12) Patent Application Publication
(10) Pub. No.: US 2012/0116135 A1
(43) Pub. Date: May 10, 2012

(54) HEAT INTEGRATED PROCESS FOR PRODUCING HIGH QUALITY PYROLYSIS OIL FROM BIOMASS

(75) Inventors: Kening Gong, Bartlesville, OK (US); Daren Einar Daugaard, Skiatook, OK (US); Alexandru Platon, Bartlesville, OK (US); Terry S. Cantu, Bartlesville, OK (US); Michael D. Wardinsky, Fulshear, TX (US)

(73) Assignee: ConocoPhillips Company, Houston, TX (US)

(21) Appl. No.: 13/290,697

(22) Filed: Nov. 7, 2011

Related U.S. Application Data
(60) Provisional application No. 61/411,531, filed on Nov. 9, 2010.

(51) Int. Cl. C07C 1/00

(52) U.S. Cl. 585/240

(57) ABSTRACT

This invention discloses a heat integrated and energy saving process for producing high quality pyrolysis oil from biomass by utilizing a torrefaction pretreatment step for biomass pyrolysis processing wherein the pretreatment step improves the quality of the pyrolysis oil by reducing acidity. This invention further utilizes the gaseous product of the torrefaction step through a combustion process for heat production and recovery.

### CROSS-REFERENCE TO RELATED APPLICATIONS

This application is a non-provisional application which claims benefit under 35 USC §119(e) to U.S. Provisional Application Ser. No. 61/411,531, filed Nov. 9, 2010, entitled “HEAT INTEGRATED PROCESS FOR PRODUCING HIGH QUALITY PYROLYSIS OIL FROM BIOMASS,” which is incorporated herein in its entirety.

### STATEMENT REGARDING FEDERALLY SPONSORED RESEARCH OR DEVELOPMENT

None.

### FIELD OF THE INVENTION

The present invention relates generally to the conversion of biomass to fuel range hydrocarbons.

### BACKGROUND OF THE INVENTION

Due to governmental legislation as mandated in the Renewable Fuels Standards (RFS), the use of renewable energy sources is becoming increasingly necessary to reduce emissions of carbon based fuels and provide alternatives to petroleum based energy and feedstock. One of the alternatives being explored is the use of biomass. Biomass is any carbon containing material derived from living or formerly living organisms, such as wood, wood waste, crops, crop waste, waste, and animal waste.

Pyrolysis is the chemical decomposition of organic materials by heating in the absence of oxygen or other reagents. Pyrolysis can be used to convert biomass (such as lignocellulosic biomass) into pyrolysis oil or so-called bio-oil. The bio-oils obtained by pyrolysis of biomass or waste have received attention recently as an alternative source of fuel.

Generally, the pyrolysis of biomass produces four primary products, namely water, “bio-oil,” also known as “pyrolysis oil,” char, and various gases (H₂, CO, CO₂, CH₄, and other light organic compounds) that do not condense, except under extreme conditions. For exemplary purposes, the pyrolysis decomposition products of wood from white spruce and poplar trees are shown in Table 1.

TABLE 1

| Source: Piskorz, J., et al. In Pyrolysis Oils | | |
|---|---|---|
| from Biomass, Soltes, E. J., Milne, T. A., | White | |
| Eds., ACS Symposium Series 376, 1988. | Spruce | Poplar |
| Moisture content, wt % | 7.0 | 3.3 |
| Particle size, μm (max) | 1000 | 590 |
| Temperature | 500 | 497 |
| Apparent residence time | 0.65 | 0.48 |
| Product Yields, wt %, m.f. | | |
| Water | 11.6 | 12.2 |
| Gas | 7.8 | 10.8 |
| Bio-char | 12.2 | 7.7 |
| Bio-oil | 66.5 | 65.7 |
| Bio-oil composition, wt %, m.f. | | |
| Saccharides | 3.3 | 2.4 |
| Anhydrosugars | 6.5 | 6.8 |
| Aldehydes | 10.1 | 14.0 |
| Furans | 0.35 | — |
| Ketones | 1.24 | 1.4 |
| Alcohols | 2.0 | 1.2 |
| Carboxylic acids | 11.0 | 8.5 |
| Water-Soluble - Total Above | 34.5 | 34.3 |
| Pyrolytic Lignin | 20.6 | 16.2 |
| Unaccounted fraction | 11.4 | 15.2 |

Fast pyrolysis is one method for the conversion of biomass to bio-oil. Fast pyrolysis is the rapid thermal decomposition of organic compounds in the absence of atmospheric or added oxygen to produce liquids, char, and gas.

Fast pyrolysis affords operation at atmospheric pressure, moderate temperatures, and with low or no water usage. Pyrolysis oil yields typically range from 50-75% mass of input biomass and are heavily feedstock dependent.

The major advantage of these fuels is that they are CO₂ neutral and contain a very low fraction of bonded sulfur and nitrogen. Thus, they contribute very little to the emission of greenhouse gases or other regulated air pollutants.

There has been a considerable effort in the past to develop pyrolysis processes for the conversion of biomass and waste to liquids for the express purpose of producing renewable liquid fuels suitable for use in boilers, gas turbines and diesel engines.

However, pyrolysis oil obtained from a biomass fast pyrolysis process is a chemically-complex mixture of compounds including water, light volatiles, and non-volatiles. Such oil is in general of relatively low quality and has a number of negative properties such as high acidity (which can lead to corrosion problems), substantial water content (usually in the range of 15% to 30%), variable viscosity, low heating values (about half that of diesel fuel), low cetane number, etc. These negative properties are related to the oxygenated compounds contained in bio-oils. The oxygen content of pyrolysis oil is approximately 45 wt %. In general, pyrolysis oil has a total acid number (TAN) value of approximately 100. The desired TAN value for transportation fuel is less than 10.

There has been a considerable effort in the past to address the high TAN problem in pyrolysis oils by post treatment or upgrading them before they are used as a fuel. Most of these treatment methods involve the removal of oxygen. Particular attention has been focused on hydrotreating using conventional petroleum catalysts, such as cobalt-molybdenum or nickel-molybdenum on alumina, to produce essentially oxygen-free naphthas. Since pyrolysis liquids typically contain between 30 to 50 wt % of oxygen, complete removal of oxygen requires a substantial consumption of hydrogen which represents a major and sometimes prohibitive cost.

Therefore, developing a new and energy saving method or process for improving quality of pyrolysis oil would be a significant contribution to the art.

### BRIEF SUMMARY OF THE DISCLOSURE

This invention discloses a heat integrated and energy saving process for producing high quality pyrolysis oil from biomass by utilizing a torrefaction pretreatment step for biomass pyrolysis processing wherein the pretreatment step improves the quality of the pyrolysis oil by reducing acidity. This invention further utilizes the gaseous product of the torrefaction step through a combustion process for heat production and recovery.

In one embodiment of the current invention, there is disclosed a process for producing a pyrolysis oil product from a biomass feedstock comprising at least the following steps: a) a step of subjecting a biomass feedstock to thermal treatment in a reactor A under a torrefaction reaction condition to produce a mixture product comprising a solid product and a gaseous product; b) a step of subjecting the solid product produced from step a) in a reactor B under a pyrolysis reaction condition to produce a product comprising pyrolysis oil product; c) a step of subjecting the gaseous product produced from step a) in a reactor C under a combustion reaction condition to produce a product comprising CO₂, H₂O and heat; and d) a step of recovering and feeding the heat produced from step c) to heat an object selected from a group consisting of the biomass feedstock, the solid product from step a), the gaseous product from step a), the pyrolysis products, the reactor A, the reactor B, and any combination thereof.

### BRIEF DESCRIPTION OF THE DRAWINGS

A more complete understanding of the present invention and benefit thereof may be acquired by referring to the following description taken in conjunction with the accompanying drawings in which:

FIG. 1 is a schematic representation of the heat generation and recovery process involved in the embodiments of the current disclosure.

### DETAILED DESCRIPTION

Embodiments of the invention disclose a heat integrated and energy saving process for producing high quality pyrolysis oil from biomass. This invention utilizes a torrefaction pretreatment step for biomass pyrolysis process wherein the pretreatment step improves the quality of the pyrolysis oil by reducing acidity. This invention further utilizes the gaseous product of the torrefaction step through a combustion process for heat production and recovery.

As used herein, the term “biomass” includes any renewable source (living or formerly living), but does not include oil, natural gas, and/or petroleum. Biomass thus includes but is not limited to wood, paper, crops, animal and plant fats, biological waste, algae, and the mixture thereof.

According to one embodiment of the invention, there is disclosed a step of subjecting a biomass feedstock to a thermal treatment in a reactor A under a torrefaction reaction condition to produce a product comprising a solid product and a gaseous product.

According to one embodiment of the invention, the torrefaction process consists of a slow heating of the biomass feedstock in an inert atmosphere to produce a solid product that has lower hemicellulose content, higher energy density, much lower moisture content (<3 wt %), and lower resistance to fracture (higher brittleness) in comparison with the initial biomass material. In addition to the solid product, the torrefaction of the biomass also produces gaseous product which may comprise CO₂, CO, H₂O, H₂, C₁/C₂/C₃ hydrocarbons, acetic acid, formic acid and other light organic compounds.

Any standard torrefaction reactor can be used to torrefy the biomass feedstock. Exemplary reactor configurations include without limitations auger reactors, ablative reactors, rotating cones, fluidized-bed reactors (e.g. circulating fluidized bed reactors), entrained-flow reactors, vacuum moving-bed reactors, transported-bed reactors, and fixed-bed reactors.

Any standard torrefaction reaction condition can be used to torrefy the biomass feedstock in the torrefaction reactor. One skilled in the art can readily select a combination of temperature, pressure, and residence time that produces a torrefied product. In some embodiments, the torrefaction reaction condition includes a temperature ranging from 180 to 350° C. with a residence time ranging from 1 minute to 24 hours. In some other embodiments, the torrefaction reaction condition includes a temperature ranging from 220 to 280° C. with a residence time ranging from 5 to 20 minutes.

A variety of pressures can be used for torrefaction, such as atmospheric pressure or ranging as high as 500 psi. Torrefaction typically operates in pressures ranging from vacuum pressures of −3 psi to above atmospheric pressure of 15 psi.

In one embodiment of the invention, torrefaction is carried out in the presence of a catalyst material selected from a group consisting of solid acid catalysts such as ZSM-5, solid base catalysts such Hydrotalcite, silica catalysts such as Diatomite, silica-alumina catalysts such as Kaolin, Group B metal oxide catalysts such as Ammonium Molybdate, pyrolytic char and any combination thereof.

In one embodiment of the invention, the torrefaction reaction is carried out in the absence of diatomic oxygen in an inert gas atmosphere such as nitrogen, argon, steam, carbon oxides, etc. In another embodiment of the invention, the torrefaction reaction is carried out in a reducing gas atmosphere (e.g., a gas atmosphere comprises carbon monoxide). Also, torrefaction may be carried out with other reactants such as hydrogen, ammonia, etc.

The torrefied biomass according to various embodiments of the invention may be added to a pyrolysis reactor for further processing. In another embodiment of the invention, the torrefied biomass is pyrolyzed in a pyrolysis reactor under a pyrolysis reaction condition to form a pyrolysis oil product.

Pyrolysis, which is the thermal decomposition of a substance into its elemental components and/or smaller molecules, is used in various methods developed for producing hydrocarbons, including but not limited to hydrocarbon fuels, from biomass. Pyrolysis requires moderate temperatures, generally greater than 325° C., such that the feed material is sufficiently decomposed to produce products which may be used as hydrocarbon building blocks.

Embodiments of the inventive process use any standard pyrolysis reactor providing sufficient heat to pyrolyze torrefied biomass feedstock, including without limitation, auger reactors, ablative reactors, a bubbling fluidized bed reactor, circulating fluidized bed, transport reactors, rotating cone pyrolyzers, vacuum pyrolyzers, and the like.

Any standard pyrolysis reaction condition can be used to pyrolyze the torrefied biomass feedstock in a pyrolysis reactor. One skilled in the art can readily select a combination of temperature, pressure, and residence time that produces a pyrolyzed product. In some embodiments, the pyrolysis reaction condition includes a temperature ranging from 375 to 700° C. with a residence time ranging from 0.01 to 200 seconds. In some other embodiments, the pyrolysis reaction condition includes a temperature ranging from 425 to 525° C. with a residence time ranging from 0.5 to 2 seconds.

A variety of pressures can be used for pyrolysis such as atmospheric pressure or greater. In some embodiments, the pyrolytic pressure ranges from vacuum conditions to 1000 psi. In other embodiments, the reaction pressure during pyrolysis can range from typical atmospheric pressure up to 300 psi.

In some embodiments, the pyrolysis reaction is carried out in the presence of a catalyst material selected from a group consisting of solid acid catalysts such as ZSM-5, solid base catalysts such Hydrotalcite, silica catalysts such as Diatomite, silica-alumina catalysts such as Kaolin, Group B metal oxide catalysts such as Ammonium Molybdate, pyrolytic char and any combination thereof.

According to various embodiments of the invention, the gaseous product from the torrefaction reactor may be sent to a combustion reactor for further processing. In some embodiments, the gaseous product is combusted in the combustion reactor under a combustion reaction condition to form a product comprising CO₂ and heat.

Any standard combustion reaction condition can be used to combust the gaseous product from the torrefaction step in a combustion reactor. One skilled in the art can readily select a combination of temperature, pressure, and residence time that produces a combustion product. In some embodiments, the combustion reaction condition includes a temperature ranging from 100 to 3000° C. In some other embodiments, the combustion reaction condition includes a temperature ranging from 400 to 1200° C.

Combustion is the burning reaction of fuel reactants with oxygen for the production of heat and light. In one embodiment, the produced gases from torrefaction are reacted with diatomic oxygen or oxygen containing air for the purpose of heat utilization during torrefaction and/or pyrolysis. Composition of the gas includes, but is not limited to, H₂, CO, CO₂, H₂O, CH₄, C₂H₂, C₂H₄, C₂H₆, C₃H₈, acetic acid, formic acid and other light organic compounds. Other gases that may also be present include O₂, N₂, and Ar as well as others.

Embodiments of the inventive process use any standard combustion reactor to combust the gaseous product from the torrefaction step, including without limitation, furnaces, combustion fluid beds, combustion fixed beds, gas turbines, kilns, gas burners, boilers, and others.

A variety of pressures can be used for combustion such as atmospheric pressure or greater. In some embodiments, the combustion pressure ranges from near atmospheric conditions to 300 psi. In other embodiments, the reaction pressure during combustion is near atmospheric pressure.

In one embodiment, the yield of gaseous product from torrefaction step is 0.1-70 wt % of raw biomass. The gaseous product comprises CO₂, CO, H₂O, H₂, C₁/C₂/C₃ hydrocarbons, acetic acid, formic acid and other light organic compounds. In one embodiment, the concentration of the CO₂ in the gaseous product ranges from 0 to 85 vol %, while the concentration of the CO in the gaseous product is in the range of 0 to 40 vol %. The concentration of H₂O in the gaseous product is in the range of 0 to 95 vol %. The total amount of H₂, C₁/C₂/C₃ hydrocarbons, acetic acid, formic acid and other light organic compounds is in the range of 0 to 70 vol %. In a different embodiment, the concentration of the CO₂ in the gaseous product ranges from 5 to 50 vol %, while the concentration of the CO in the gaseous product is in the range of 0-30 vol %. The concentration of H₂O in the gaseous product is in the range of 30-80 vol %. The total amount of H₂, C₁/C₂/C₃ hydrocarbons, acetic acid, formic acid and other light organic compounds is normally below 50 vol %.

The gaseous product of torrefaction can not be directly released to the atmosphere mainly due to the high concentration of CO and organic compounds in the stream. One embodiment of the current invention converts (via e.g. combustion) CO and organic compounds into CO₂ which can then be directly released to the atmosphere without violating environmental regulations (e.g., CO emission specification). Depending on local emission regulations, some extra treatment might need to be placed downstream of the combustion reactor.

Since the reactions of torrefaction and pyrolysis are endothermic, to maintain normal operation and desired product quality, heat must be supplied constantly to these two reactions. According to one embodiment of the invention, the heat produced from torrefaction gaseous product combustion may be utilized for the torrefaction and/or pyrolysis reactions. The produced gas can initially be combusted in a combustion reactor providing heat for torrefaction and/or pyrolysis reactions through a heat carrier, such as solid catalyst, sand, steam, and flue gas. For example, the produced torrefaction gaseous products can be combusted in a fluidized bed or fast transport bed containing solid catalyst or other solid particles. The heated catalyst or solid particles can then be used to provide heat for the endothermic torrefaction and/or pyrolysis reactions. According to one embodiment of the invention, the heat from combustion of torrefaction gaseous products can be utilized to pre-heat the feedstocks of the torrefaction and/or pyrolysis reactors, or to directly heat the reactors in order to maintain reaction temperature. The heat produced from combustion of torrefaction gaseous products can also be recovered to generate some products, such as process steam and electricity. These process steam and electricity products not only may be used to provide heat for the torrefaction and/or pyrolysis processes, but also may be utilized for other processes.

According to one embodiment of the invention, the torrefaction and/or pyrolysis process described above is carried out in the presence of a carrier gas stream, including but not limited N₂, He, CO₂, and Ar, and the heat from combustion of torrefaction gaseous products can be utilized to pre-heat the biomass feedstocks, the carrier gas stream, or the reactors directly in order to maintain reaction temperature.

The final pyrolysis oil product obtained according to some embodiments of the present invention has a TAN number between 80 and 200. The pyrolysis oil product obtained according to some other embodiments of the present invention has a TAN number between less than 20 and 50.

The following examples of certain embodiments of the invention are given. Each example is provided by way of explanation of the invention, one of many embodiments of the invention, and the following examples should not be read to limit, or define, the scope of the invention.

### EXAMPLE 1

The comparison study of the process of torrefaction prior to pyrolysis has been performed in a micropyrolysis unit. The reactions were carried out at torrefaction temperatures ranging from 179 to 321° C. and pyrolysis temperatures ranging from 379 to 521° C. with no catalyst loading. In addition, a wide variety of biomass was tested including red oak, switchgrass, miscanthus, and corn stover pellets. Comparative pyrolysis tests were run without the torrefaction pretreatment at the same pyrolysis temperatures.

### Result:

The experimental results indicating the reduction of acetic acid in the pyrolysis product due to torrefaction are shown as follows:

TABLE I
Average Acetic Acid Yield.

| | Pyrolysis | | Torrefaction - Pyrolysis | | | |
|---|---|---|---|---|---|---|
| | Yield¹, | | Yield¹, | | Reduction³ | |
| Biomass | wt-% | Concentration², % | wt-% | Concentration², % | Yield, % | Concentration, % |
| Oak | 8.76 | 5.38 | 6.29 | 4.40 | 28.2 | 18.1 |
| Switchgrass | 4.64 | 4.79 | 3.07 | 3.96 | 34.0 | 17.3 |
| Miscanthus | 3.75 | 6.25 | 2.35 | 4.41 | 37.2 | 29.4 |
| Corn | 1.89 | 5.41 | 0.74 | 3.29 | 60.7 | 39.3 |
| Stover | | | | | | |
| Pellets | | | | | | |

¹Mass of acetic acid over mass of biomass
²Acetic acid peak area over total peak area by GC/MS
³Torrefaction - pyrolysis acetic acid level relative to pyrolysis acetic acid level

The result above shows that the acetic acid concentration in pyrolysis oil products was reduced by 18 to 39% with this pretreatment, compared to that from un-torrefied biomass. The resulting pyrolysis oil would have a similar reduction in TAN (total acid number) value as ˜80% of the TAN is due to acetic acid in pyrolysis oils.

### EXAMPLE 2

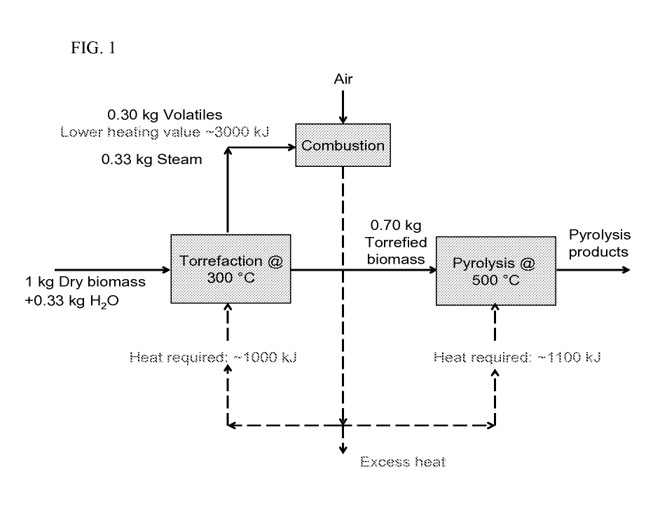

As illustrated in FIG. 1, the theoretical heat available from the combustion process according to the current invention is calculated based on the assumption that 1 kg of dry biomass with 25 wt % of moisture content is torrefied at 300° C., the volatile product yield is 30% (dry biomass basis), the volatiles include CO, CO₂, H₂O (produced from torrefaction reaction), H₂, methane, ethane, acetic acid, formic acid, and other organic materials. The calculated lower heating value (LHV) of the 0.3 kg volatiles/gases is approximately 3000 KJ. The heat produced from combustion of these volatiles is then utilized by torrefaction and/or pyrolysis steps, which require 1000 KJ, and/or 1100 KJ, respectively.

### Discussion:

As discussed above, the pyrolysis oil obtained from biomass fast pyrolysis process is of relatively low quality. In general, pyrolysis oil has TAN value of approximately 100. The desired TAN value for transportation fuel is less than 10.

The results above show that using torrefied biomass as a pretreated feed for pyrolysis helps reduce TAN (total acid number) of the pyrolysis oil product. The pretreatment by torrefaction according to the current invention helps to significantly reduce the TAN value of the pyrolysis oil product by 25%. This is mainly attributed to the release of acetic acid in the torrefaction step.

The step of torrefaction and a heat generation and recovery step may be easily integrated with the pyrolysis step. The biomass pretreatment by torrefaction improves the biomass feed quality of pyrolysis step and therefore resulting in higher quality of pyrolysis oil product including low TAN value. The heat generation and recovery step convert the gaseous product from the torrefaction step into heat which can be recovered and utilized for torrefaction and/or pyrolysis. The heat produced as described can also be recovered to produce process steam and electricity. Therefore, this heat-integrated process according to the current invention helps to improve the pyrolysis oil produce and reduce the energy consumption and operating costs.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise. It should also be noted that, as used in this specification and the appended claims, the phrase “configured” describes a system, apparatus, or other structure that is constructed or configured to perform a particular task or adopt a particular configuration to. The phrase “configured” can be used interchangeably with other similar phrases such as arranged and configured, constructed and arranged, constructed, manufactured and arranged, and the like.

Although the systems and processes described herein have been described in detail, it should be understood that various changes, substitutions, and alterations can be made without departing from the spirit and scope of the invention as defined by the following claims. Those skilled in the art may be able to study the preferred embodiments and identify other ways to practice the invention that are not exactly as described herein. It is the intent of the inventors that variations and equivalents of the invention are within the scope of the claims whiles the description, abstract and drawings are not to be used to limit the scope of the invention. The invention is specifically intended to be as broad as the claims below and their equivalents. In closing, it should be noted that each and every claim below is hereby incorporated into this detailed description or specification as an additional embodiments of the present invention.

That which is claimed:

1. A process for producing a pyrolysis oil product from a biomass feedstock comprising at least the following steps:
a) a step of subjecting a biomass feedstock to thermal treatment in a reactor A under a torrefaction reaction condition to produce a mixture product comprising a solid product and a gaseous product;
b) a step of subjecting said solid product in a reactor B under a pyrolysis reaction condition to produce a product comprising pyrolysis oil product;
c) a step of subjecting said gaseous product in a reactor C under a combustion reaction condition to produce a product comprising CO₂, H₂O and heat; and
d) a step of recovering and feeding said heat from step c) to heat an object selected from a group consisting of said biomass feedstock, said solid product from step a), said gaseous product from step a), said pyrolysis products from step b), said reactor A, said reactor B, and any combination thereof

2. The process according to claim 1, wherein said step a) or step b) are carried out in the presence of a carrier gas stream, and wherein said object further consisting of said carrier gas stream.

3. The process according to claim 2, wherein said carrier gas stream is selected from a group consisting N₂, He, CO₂, and Ar.

4. The process according to claim 1, wherein said step c) is carried out in the presence of a heat carrier, wherein said heat carrier is selected from a group consisting of solid catalysts, solid particles, steam and flue gas.

5. The process according to claim 1,
wherein said step c) is carried out in fluidized bed or fast transport bed in the presence of catalysts or solid particles whereby heated catalysts and heat solid particles may obtained; and
wherein said heated catalysts or said heated particles are fed to an object selected from a group consisting of said biomass feedstock, said solid product from step a), said gaseous product from step a), said pyrolysis products from step b), said reactor A, said reactor B, and any combination thereof

6. The process according to claim 1, further comprises steps of
i) generating a process steam from said heat after step c); and
ii) feeding said process steam to an object selected from a group consisting of said biomass feedstock, said solid product from step a), said gaseous product from step a), said pyrolysis products from step b), said reactor A, said reactor B, and any combination thereof

7. The process according to claim 1,
wherein said torrefaction reaction condition includes a temperature ranging from 180 to 350° C., a pressure ranging from atmospheric to 500 psig, and a residence time ranging from 1 minute to 24 hours;
wherein said pyrolysis reaction condition includes a temperature ranging from 375 to 700° C., a pressure ranging from vacuum condition to 1000 psig, and a residence time ranging from 0.01 to 200 seconds;
wherein said combustion reaction condition includes a temperature ranging from 100 to 3000° C., a pressure ranging from near atmospheric pressure to 300 psi, with a residence time ranging from 0.01 millisecond to 30 minutes; and
wherein said pyrolysis oil product has a total acid number (TAN) between 80 and 200.

8. The process according to claim 1,
wherein said torrefaction reaction condition includes a temperature ranging from 220 to 240° C., a pressures ranging from vacuum pressures of −3 psig to above atmospheric pressure of 15 psig, and a residence time ranging from 5 to 20 minutes;
wherein said pyrolysis reaction condition includes a temperature ranging from 425 to 525° C., a pressure ranging from atmospheric pressure to 300 psi, and a residence time ranging from 0.5 to 2 seconds;
wherein said combustion reaction condition includes a temperature ranging from 400 to 1200° C., a pressure of near atmospheric pressure, and a residence time ranging from 0.1 millisecond to 30 seconds; and
wherein said pyrolysis oil product has a TAN number between 20 and 50.

9. The process according to claim 1, wherein said torrefaction reaction is carried out in reactor A selected from a group consisting of augers reactors, ablative reactors, rotating cones, fluidized-bed reactors, circulating fluidized bed reactors, entrained-flow reactors, vacuum moving-bed reactors, transported-bed reactors, and fixed-bed reactors.

10. The process according to claim 1, wherein said pyrolysis reaction is carried out in reactor B selected from a group consisting of auger reactors, ablative reactors, a bubbling fluidized bed reactor, circulating fluidized beds/transport reactor, rotating cone pyrolyzer, and vacuum pyrolyzer.

11. The process according to claim 1, wherein said combustion reaction is carried out in reactor C selected from a group consisting of furnace, combustion fluid beds, combustion fixed beds, gas turbines, kilns, gas burners, and boilers.

12. The process according to claim 1, wherein said torrefaction reaction is carried out in the presence of a catalytic material selected from a group consisting solid acid catalysts, solid base catalysts, silica catalysts, silica-alumina catalysts, Group B metal oxide catalysts, pyrolytic char and any combination thereof

13. The process according to claim 12, wherein said solid acid catalyst is ZSM-5, said solid base catalyst is Hydrotalcite, said silica catalyst is Diatomite, said silica-alumina catalyst is Kaolin, and said Group B metal oxide catalyst is Ammonium Molybdate.

14. The process according to claim 1, wherein said pyrolysis reaction is carried out in the presence of a catalyst material selected from a group consisting solid acid catalysts, solid base catalysts, silica catalysts, silica-alumina catalysts, Group B metal oxide catalysts, pyrolytic char and any combination thereof

15. The process according to claim 14, wherein said solid acid catalyst is ZSM-5, said solid base catalyst is Hydrotalcite, said silica catalyst is Diatomite, said silica-alumina catalyst is Kaolin, and said Group B metal oxide catalyst is Ammonium Molybdate.

16. The process according to claim 1, wherein said biomass feedstock is selected from the group consisting of, wood, paper, crops, animal and plant fats, biological waste, algae and mixture thereof.

17. The process according to claim 1, wherein said solid product comprises torrefied biomass feedstock.

18. The process according to claim 1, wherein said gaseous product in step a) comprises CO₂, CO, H₂O, H₂, C₁/C₂/C₃ hydrocarbons, acetic acid, formic acid and other light organic compounds.

19. The process according to claim 18,
wherein the concentration of said CO₂ in said gaseous product ranges from 5 to 50 vol %;
wherein the concentration of said CO in said gaseous product ranges from 0 to 30 vol %;
wherein the concentration of said H₂O in said gaseous product ranges from 30 to 80%, and
wherein the concentration of the total amount of H₂, C1/C2/C3 hydrocarbons, acetic acid, formic acid and other light organic compounds in said gaseous product ranges from 0 to 50 vol %.

20. The process according to claim 18,
wherein the concentration of said CO₂ in said gaseous product ranges from 0 to 85 vol %;
wherein the concentration of said CO in said gaseous product ranges from 0 to 40 vol %;
wherein the concentration of said H₂O in said gaseous product ranges from 0 to 95%, and
wherein the concentration of the total amount of H₂, C1/C2/C3 hydrocarbons, acetic acid, formic acid and other light organic compounds in said gaseous product ranges from 0 to 70 vol %.

21. The process according to claim 1, wherein said torrefaction reaction is carried out in the absence of diatomic oxygen in an inert gas atmosphere selected from a group consisting of nitrogen, argon, steam, and carbon oxides.

22. The process according to claim 1, wherein said torrefaction reaction is carried out in a reducing gas atmosphere.

23. The process according to claim 1, wherein said torrefaction reaction is carried out in a gas atmosphere comprising carbon monoxide.

24. The process according to claim 1, wherein said torrefaction reaction is carried out with a reactant selected from a group consisting of hydrogen and ammonia.
